(19) United States
(12) Patent Application Publication
(10) Pub. No.: US 2023/0018446 A1
(43) Pub. Date: Jan. 19, 2023

(54) AIR PURIFICATION SYSTEMS AND METHODS FOR VACUUM CLEANERS

(71) Applicant: OHC IP Holdings, LLC, Eden Prairie, MN (US)

(72) Inventors: Michael O'Shaughnessy, Eden Prairie, MN (US); James Mitlyng, Eagan, MN (US); Rosie Colacino, Hopkins, MN (US); Logan Oglesby, Savage, MN (US); Helai Li, Maple Grove, MN (US); Jason R. Geidel, West Bend, WI (US); Michael D. Peters, West Bend, WI (US); Peter J. Heinen, Jackson, WI (US); Brady K. Watry, Fredonia, WI (US)

(21) Appl. No.: 17/813,301

(22) Filed: Jul. 18, 2022

Related U.S. Application Data
(60) Provisional application No. 63/223,016, filed on Jul. 18, 2021.

(51) Int. Cl. B01D 46/00; A47L 9/30; A47L 9/12; A47L 9/28; B01D 46/10; B01D 53/04; A61L 9/20

(52) U.S. Cl. CPC B01D 46/0028; A47L 9/30; A47L 9/122; A47L 9/2836; B01D 46/0038; B01D 46/10; B01D 53/0407; A61L 9/20; B01D 2253/102; B01D 2257/708; B01D 2279/55; A61L 2209/14; A61L 2209/12

(57) ABSTRACT

Disclosed are devices, systems and methods for air purification in an airflow tract of vacuum cleaners. In some aspects, an air purification system for a vacuum cleaner comprises an ultraviolet (UV) light unit disposed in a first location within the vacuum cleaner along an airflow pathway, configured to emit UV light at air containing particles including dust, dirt, and microbes while the air containing the particles is flowing in the airflow pathway where the UV light unit is disposed; and a particle filter unit disposed in a second location within the vacuum cleaner along the airflow pathway, the particle filter unit comprising one or both of a high-efficiency particulate air (HEPA) filter and an active carbon filter.

### CROSS-REFERENCE TO RELATED APPLICATIONS

This patent document claims priority to and benefits of U.S. Provisional Patent Application No. 63/223,016, tilted “AIR PURIFICATION SYSTEMS AND METHODS FOR VACUUM CLEANERS” and filed on Jul. 18, 2021. The entire content of the aforementioned patent application is incorporated by reference as part of the disclosure of this patent document.

### TECHNICAL FIELD

This patent document relates to vacuum cleaners.

### BACKGROUND

Developments in vacuum cleaner technology has led to an evolution of vacuum cleaner products, including from upright vacuum cleaners to handheld vacuum cleaners and from using one time use paper bags to collect debris to using a reusable container that can dispose the collected debris. Vacuum cleaner products can remove debris from a surface using air suction, which may vary in efficacy and efficiency due to a variety of factors.

### SUMMARY

Disclosed are devices, systems and methods for air purification in an airflow tract of vacuum cleaners.

In some aspects, an air purification system for a vacuum cleaner includes an ultraviolet (UV) light unit disposed in a first location within the vacuum cleaner along an airflow pathway, the UV light unit comprising a housing and one or more UV light emitters coupled to the housing, the UV light unit configured to emit UV light at air containing particles including dust, dirt, and microbes while the air containing the particles is flowing in the airflow pathway where the UV light unit is disposed, wherein emitted UV light is able to harm biological materials of the microbes to sterilize the air; and a particle filter unit disposed in a second location within the vacuum cleaner after the first location along the airflow pathway, the particle filter unit comprising one or both of a high-efficiency particulate air (HEPA) filter and an active carbon filter, the HEPA filter including a porous material to prevent at least some of the particles having a size greater than a pore size of the porous material from passing through the HEPA filter, and the active carbon filter including a securement structure that couples an activated carbon material having a chemically-reactive surface capable of filtering molecules within the air contacting the active carbon filter by facilitating chemical reactions with the molecules to remove from the air.

In some aspects, an air purification system for a vacuum cleaner includes an ultraviolet (UV) light unit disposed in a first location within the vacuum cleaner along an airflow pathway from a suction inlet to an exhaust outlet, the UV light unit comprising a first chamber and a second chamber and a first set of one or more UV light emitters and a second set of one or more UV light emitters positioned within the first chamber and the second chamber, respectively, wherein the UV light unit is configured to emit UV light via the one or more UV light emitters within the first chamber and the second chamber at air containing microbes while the air containing the microbes is flowing in the first chamber and the second chamber, wherein emitted UV light is able to harm biological materials of the microbes to sterilize the air; and a particle filter unit disposed in a second location within the vacuum cleaner positioned before the first location along the airflow pathway, the particle filter unit comprising one or both of a high-efficiency particulate air (HEPA) filter and an active carbon filter.

The subject matter described in this patent document can be implemented in specific ways that provide one or more of the following features.

### BRIEF DESCRIPTION OF THE DRAWINGS

FIG. 1 shows a partial schematic block diagram of a vacuum cleaner.

FIGS. 2A-2D show diagrams of an example embodiment of a vacuum cleaner that includes an air purification system in accordance with the present technology.

FIGS. 2E and 2F show diagrams of example embodiments of a scent system for a vacuum cleaner, in accordance with the present technology.

FIG. 3 shows a diagram of a vacuum cleaner that includes an example embodiment of the air purification system, in accordance with the present technology.

FIGS. 4A and 4B show diagrams of an example embodiment of the UV light unit of an example air purification system, in accordance with the present technology.

FIG. 5 shows a diagram illustrating locations of a vacuum cleaner employing an example embodiment of a sealed system to prevent leaks of dirty air, in accordance with the present technology.

FIG. 6 shows a diagram illustrating an example embodiment of a particulate filter unit that incorporates an example embodiment of the sealed system of FIG. 5.

FIG. 7A shows a diagram illustrating an example embodiment of a low airflow sensing system, in accordance with the present technology.

FIG. 7B shows a diagram illustrating an example embodiment of a low airflow sensing system positioned within a debris collection canister to indicate whether the canister is full, in accordance with the present technology.

FIG. 8A shows a diagram illustrating an example embodiment of a hybrid upright floor-rolling—detachable lift-away vacuum cleaner, in accordance with the present technology.

FIG. 8B shows a diagram depicting an example embodiment of a cordless, battery-powered configuration for the hybrid upright floor-rolling—detachable lift-away vacuum cleaner of FIG. 8A.

### DETAILED DESCRIPTION

Generally, a vacuum cleaner creates a suction force pulling in air, which causes dirt, dust, microbes, allergens, and other particles of various sizes to be dispersed within the sucked air into the vacuum cleaner. In order to create the suction force, the air that is forced in is also forced out of the vacuum cleaner, but ideally without the dirt, dust, microbes, allergens, and other particles along with the expelled air. Conventionally, the vacuum cleaner traps particles within a container (e.g., a disposable bag or bagless canister or chamber) based on filter(s) that allow the forced air to pass through but prevent the particles from passing. The filter is typically a porous material, where the particles in the forced air that are larger than the filter's pore size are trapped; however, particles that are smaller than the filter's pore size pass through with the expelled air, recycling back into the outer environment of the vacuum.

Commonly, the small-sized particles that are expelled with the exhaust air from the vacuum cleaner contain harmful microbes, which were previously settled prior to the suction, but subsequently dispersed back into air that may be breathed in by those in the environment. Therefore, it would be beneficial to sterilize the air that is cycled within a vacuum cleaner. Yet, conventional vacuum cleaners do not provide sufficient means to both effectively sterilize and purify the air, and consequently unsterile, microbe-contaminated air becomes recycled into the environment during and after use of conventional vacuum cleaners.

Disclosed are devices, systems and methods for air purification, which may be implemented within a vacuum cleaner.

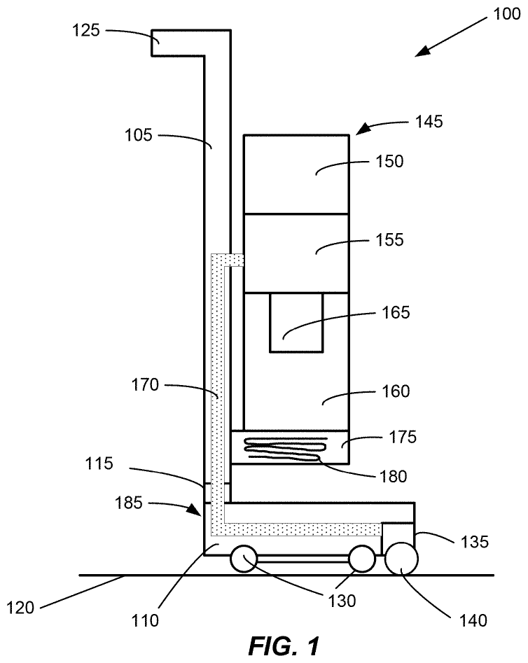

FIG. 1 illustrates a partial schematic block diagram of a vacuum cleaner 100, configured in accordance with embodiments of the present technology. In some embodiments, the vacuum cleaner 100 includes an upper frame 105 connected to a lower assembly 110 via a joint 115. The joint 115 can facilitate movement of the upper frame 105 relative to the lower assembly 110 while the upper frame 105 and the lower assembly 110 are connected together via the joint 115. For example, the upper frame 105 can pivot, rotate, or otherwise move relative to the lower assembly 110 to facilitate a user's operation of the vacuum cleaner 100, such as pushing and steering the lower assembly 110 along a surface 120 using the upper frame 105.

Accordingly, the upper frame 105 can support a handle portion 125 positioned for a user to grasp during operation of the vacuum cleaner 100, and the lower assembly 110 can optionally carry one or more motility units 130 (such as wheels or tracks) to facilitate travel of the lower assembly 110 along the surface 120. In some embodiments, the motility units 130 can include powered motility units, such as motorized wheels. In other embodiments, the motility units 130 can be unpowered (such as freewheeling or otherwise freely movable), except for the pushing force provided by a user upon the vacuum cleaner 100.

In some embodiments, the lower assembly 110 carries a suction input 135, which receives a suction airflow to pull debris (such as waste or particles) from the surface 120. To aid in releasing debris from the surface 120, the lower assembly 110 can also carry a brush 140. The brush 140 can agitate debris on the surface 120 to facilitate release of the debris and suction of the debris into the suction input 135. In some embodiments, the brush 140 is motorized such that it rotates against the surface 120. In some embodiments, the suction input 135 includes a main suction input and one or more auxiliary suction inputs.

In some embodiments, the vacuum cleaner 100 includes one or more suction and collection components 145 to facilitate providing suction to the suction input 135 and collection of debris from the suction input 135. For example, the suction and collection components 145 can include: a suction drive unit 150 operatively connected to the suction input 135 to provide the suction airflow; a filtration unit 155 operatively connected between the suction input 135 and the suction drive unit 150 to remove debris from the suction airflow passing from the suction input 135 to the suction drive unit 150; and/or a debris collection chamber 160 operatively connected to the filtration unit 155 to collect debris that is filtered from the suction airflow. In some embodiments, the suction and collection components 145 can include a debris passage 165 between the filtration unit 155 and the debris collection chamber 160 to facilitate passage of debris from the filtration unit 155 to the debris collection chamber 160. The suction and collection components 145 can be connected to the suction input 135 via an airflow pathway 170. For example, the airflow pathway 170 can connect the suction input 135 to the filtration unit 155. In some embodiments, the suction drive unit 150 includes a motor, fan, or other suitable source of airflow suction. The debris collection chamber 160 can be removable and replaceable from the vacuum cleaner 100 to facilitate emptying the debris collection chamber 160.

In some embodiments, some or all of the suction and collection components 145 can be carried by the upper frame 105. For example, the upper frame 105 can include a base 175 positioned to support some or all of the suction and collection components 145. In some embodiments, the base 175 can store a power cord 180 for connecting the vacuum cleaner 100 to an external power source. In some embodiments, the base 175 can house electronic components, including but not limited to a power converter (e.g., AC/DC conversion) and/or a power supply (e.g., a battery).

In some embodiments, the joint 115 can facilitate separation of the upper frame 105 from the lower assembly 110. In such embodiments, some or all of the suction and collection components 145 can be carried by the lower assembly 110, such that the lower assembly 110 and the suction and collection components 145 can form a lower vacuum unit 185 that can be operable independently of the upper frame 105 and the handle portion 125. In some embodiments, the airflow pathway 170 can include a hose that is detachable from the lower assembly 110, in which the detachable end of the hose can allow for attachment pieces (not shown), e.g., such as a pet hair brush, dusty brush, crevice tool, etc., to attach to the hose's detachable end and allow a user to vacuum clean surfaces difficult or otherwise unreachable by the lower assembly 110.

Although FIG. 1 illustrates a vacuum cleaner 100 configured in accordance with some embodiments of the present technology, in other embodiments, the vacuum cleaner 100 can be configured in other ways.

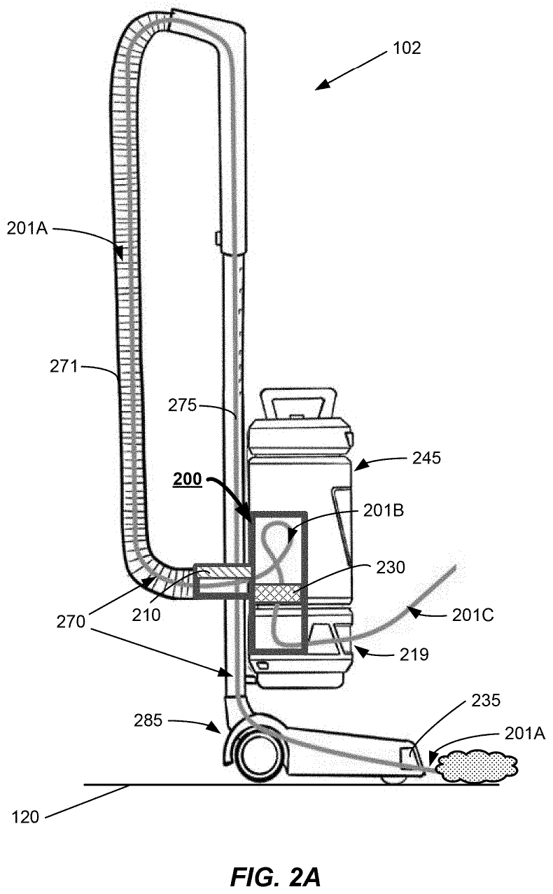

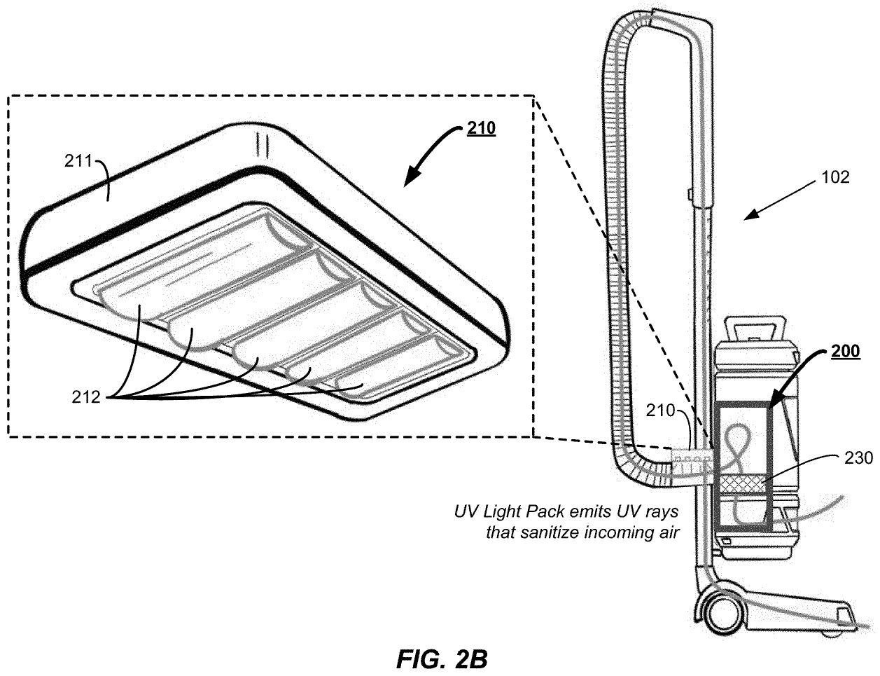

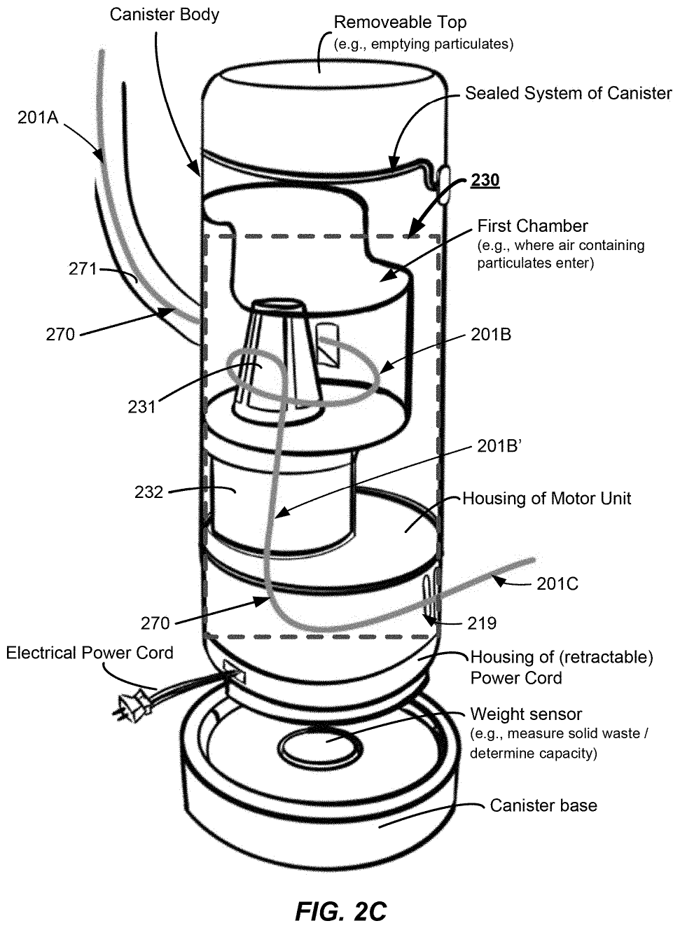

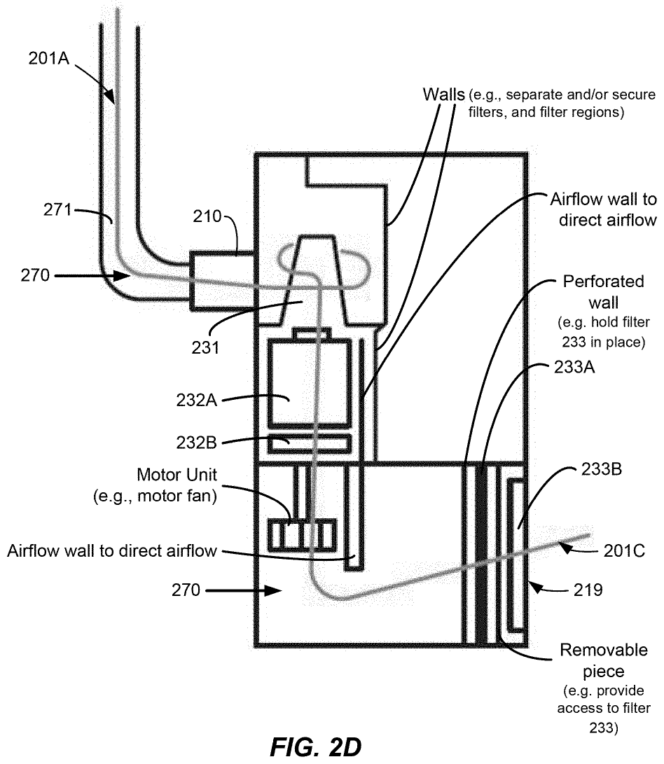

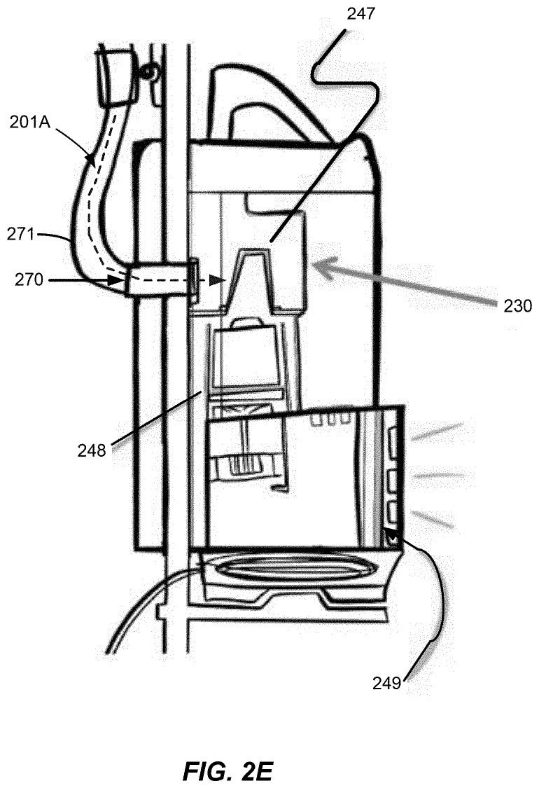

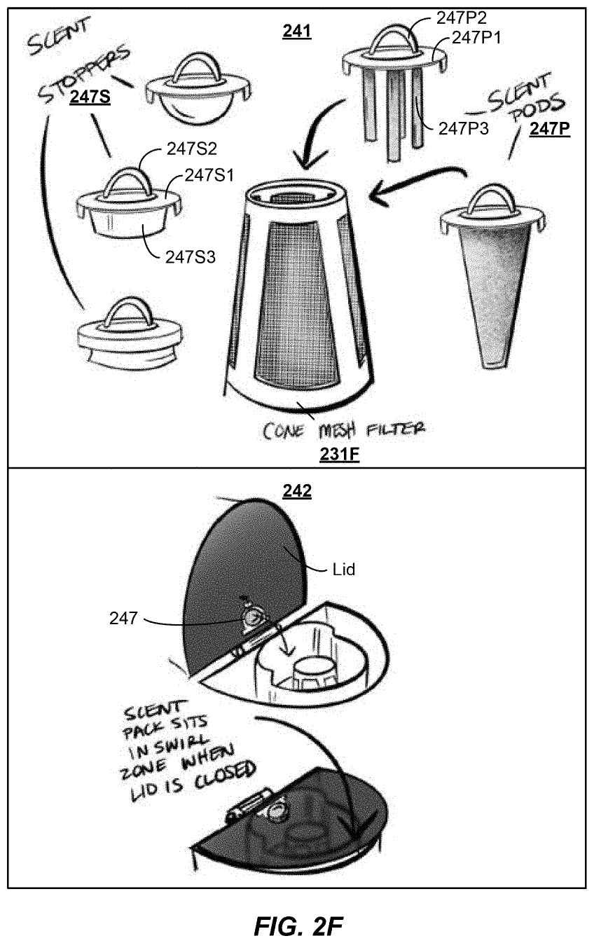

FIG. 2A shows a diagram of an example embodiment of the vacuum cleaner 100, shown as vacuum cleaner 102, that includes an air purification system 200 in accordance with the present technology. The air purification system 200 is specially integrated in subunits or components of the vacuum cleaner 100 to produce a sterilized and purified output air 201C from an exhaust 219 at a terminus of an example embodiment of the airflow pathway 170, shown as airflow pathway 270. As illustrated in the diagram, an input air 201A containing dirt, dust, microbes, etc. (collectively referred to as “filthy air” 201A) is sucked into a suction input 235 of the vacuum cleaner 102 and travels through the airflow pathway 270, illustrated via flow path line 275. In this example, the filthy air 201A is collected at the suction input 235 of a lower assembly 285 from which it travels through a hose 271 of the airflow pathway 270 to suction and collection assembly 245, which can include a suction drive unit, a filtration unit, and/or a debris collection chamber (like in the example of the vacuum cleaner 100 shown in FIG. 1). While traveling through the airflow pathway 270, the filthy air 201A is passed through the air purification system 200, which can be positioned in one or more components of the vacuum cleaner 100 and configured in one or more housing structures, which can be connected or separated in various example embodiments.

In some embodiments, for example, the air purification system 200 includes a UV light unit 210 that includes at least one UV light emitter configured to emit ultraviolet (UV) light at the filthy air 201A in the airflow pathway 270 where the UV light unit 210 is disposed. In some implementations, the UV light unit 210 first sterilizes the filthy air 201A, e.g., prior to filtration of particulates, by emittance of the UV light that disrupts cellular and/or protein structures of microbes (e.g., bacteria, protists, fungi, viruses, etc.) for harming and/or killing the microbes. In implementations, for example, the UV light unit 210 of the air purification system 200 is able to at least partially sterilize the filthy air 201A to become sterilized, particulate-containing air 201B. The sterilized particulate-containing air 201B is then passed, via the suction of the vacuum cleaner 102, to the particulate filter unit 230, which comprises two or more filters to remove particulates (including the dust, dirt, microbes (having just been sterilized), etc.) from the air flow.

FIG. 2B shows a diagram illustrating an example embodiment of the UV light unit 210, which is disposed in a region of the airflow pathway 270 that feeds the suction and collection assembly 245. The example UV light unit 210 includes a housing or frame structure 211 that secures one or more UV light emitters 212 to emit UV light as rays to sterilize air containing microbes (of the filthy air 201A) as it flows past through a UV light emission zone within the airflow pathway 270. The example of FIG. 2B shows one example of the UV light assembly 210 proximate the interface between the hose 271 of the airflow pathway 270 and the input of the suction and collection assembly 245; yet, the UV light assembly 210 can include a plurality of UV light assemblies that are positioned in other regions of the airflow pathway, prior to and after the interface between the hose 271 and the input of the suction and collection assembly 245.

In some implementations of the air purification system 200, the UV light unit 210 is configured to emit UV light in the ultraviolet C (UV-C) sub-spectrum (e.g., 100 to 280 nm wavelength) of the ultraviolet spectrum. In some embodiments, for example, one or more of the UV light emitters 212 can include a UV lamp (e.g., shortwave UV fluorescent lamp, or incandescent UV halogen lamp), a UV light-emitting diode (LED), or a UV laser (e.g., UV gas laser, UV solid-state laser, or laser diode). In some embodiments, for example, the UV light unit 210 can be configured to emit pulsed UV light, in which a series of high magnitude pulse light rays across a broad portion of the UV spectrum, e.g., including UV-C, is emitted.

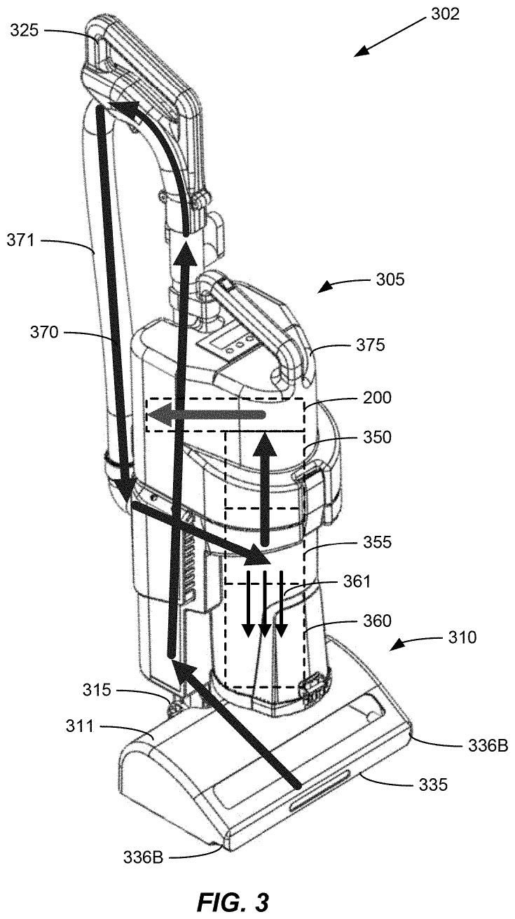

Ultraviolet radiation can damage and kill living tissues by damaging cellular DNA, for example. As such, the UV light unit 210 is configured within a shielded compartment inside the vacuum cleaner 102 so that the UV energy (e.g., UV-C waves) emitted by the one or more UV light emitters 212 do not propagate out of the vacuum cleaner 102 to incidentally risk exposure to living organisms outside the air purification system 200. For instance, some conventional vacuum cleaner products use various wavelengths within the range of UV light to sanitize and disinfect surfaces by directing the UV light directly on the surface to be cleaned, e.g., to kill germs or microbes like bacteria and viruses. However, such techniques risk harm by UV light exposure outside the vacuum. In some example embodiments of the air purification system 200, the housing or frame structure 211 of the UV light unit 210 can include one or more surrounding walls in the location or compartment where the UV light unit 210 is disposed along the airflow pathway 270, such that the UV light unit 210 can contain the emitted UV light (e.g., including the most energetic range of UV waves: UV-C waves) internally within the vacuum cleaner 102. In some embodiments, for example, the UV light unit 210 is positioned pre-debris filtration (as illustrated in the example shown in FIGS. 2A, 2B and 2D), which can prevent harmful microbes from existing in the debris collection and thereby potentially re-entering the living space; whereas in some embodiments, the UV light unit 210 is positioned post-debris filtration (as illustrated in the example shown later in FIGS. 3A and 3B), such that the vacuum's exhaust air is sanitized to prevent harmful miniscule germs from re-entering the living space through the exhaust air; whereas in some embodiments, for example, the vacuum cleaner 102 can include at least two UV light units 210 where one is positioned pre-debris filtration and another is positioned post-debris filtration. In implementations, for example, the UV light unit 210 can help to ensure that, even if any germ particle could pass through the vacuum cleaner's high-efficiency filters and into the exhaust stream, it will be completely neutralized (e.g., bio-inactivated).

In some implementations of the air purification system 200, the UV light unit 210 can be configured to be in electrical communication with an electronics unit housed in the vacuum cleaner 102, which can include a power converter (e.g., AC/DC conversion), power regulator, and/or a power supply (e.g., a battery) to supply the appropriate electrical current to the UV light emitter(s) 212. In some embodiments, for example, the UV light unit 210 includes a control unit, comprising a processor and memory, to provide the control logic in operations of the UV light emitter(s) 212. For example, the control unit can regulate the intensity, pulse frequency, and/or duration of the emitted UV light. For example, in some implementations, the intensity of UV energy (e.g., UV-C radiation) can be adjusted by an end user, e.g., based on predetermined values or limits (which can be represented as levels), to increase or decrease the intensity and/or pulse frequency of the UV light, e.g., where levels can be predetermined based on calculated values of the parameters known to eradicate a multitude of common airborne pathogens, including SARS-CoV-2. Such predetermined values or limits associated with adjustable UV energy levels can account for the volumetric unit of air and/or flow rate in the UV light unit 210 in accordance with the intensity and/or pulse frequency of the UV radiation, so that the harmful microbes in the air flow through the UV light unit 210 are sufficiently exposed to the emitted UV light temporally and spatially.

FIG. 2C shows a perspective view diagram illustrating aspects of an example embodiment of the particulate filter unit 230, which is disposed in suction and collection assembly 245 along the airflow pathway 270. As shown by the example of FIG. 2C, the suction and collection unit 245 includes a canister body having a removeable top (or partially removeable top) to allow a user to access an interior of the canister body. The removeable top and canister body are sealable through a sealing system, which can include a material that contacts an end of the removeable top and/or canister body to facilitate a seal (e.g., an airtight seal).

After the filthy air 201A is sterilized or at least partially sterilized by the UV light unit 210 to produce the sterilized particulate-containing air 201B, e.g., prior to entering a first chamber of the canister body in this embodiment, the sterilized particulate-containing air 201B is pulled by suction through a first filter 231 of the in suction and collection assembly 245, prior to filtering by the particulate filter unit 230. In some embodiments, for example, the first filter 231 includes a mesh filter to exclude the sterilized particulates of a certain size. For example, the first filter 231 can include a mesh having 10 to 100 pores per inch, and/or a pore size in a range of 0.005 inch to 0.1 inch, which prevents particulates of a size greater than about 125 μm (or greater) from passing through the first filter 231. In some embodiments, for example, the mesh filter can be configured as an inverted cone that is detachable from a base of the first chamber, e.g., to allow for easy cleaning of the filter by a user.

After the sterilized particulate-containing air 201B has been filtered by the first filter 231, the filtered-sterilized particulate-containing air 201B′ enters a subsequent region of the suction and collection assembly 245 where additional subunits of the air purification system 200 are integrated, including the particulate filter unit 230 (and in some embodiments, additional UV light emitters) to purify the air as it exits the exhaust 219 to flow out sterilized and purified output air 201C.

FIG. 2D shows a side view diagram illustrating an example embodiment of the particulate filter unit 230, integrated in the suction and collection assembly 245 along the airflow pathway 270. In some embodiments, for example, the particulate filter unit 230 includes a second filter 232, comprising one or more secondary particulate filters 232A and/or 232B, which is positioned after the first filter 231 along the airflow pathway 270. The second filter 232 is configured to filter particulates among the sterilized particulate-containing air 201B that are smaller than the smallest pore size of the first filter 231, but larger than one or more pore sizes of the secondary particulate filters 232A and/or 232B. In embodiments including the secondary particulate filter 232A, for example, the secondary particulate filter 232A can include a foam particle filter, e.g., polyurethane material, having 10 to 100 pores per inch, and/or a pore size in a range of 0.004 inch to 0.1 inch, which prevents particulates of a size greater than about 100 μm (or greater) from passing through the secondary particulate filter 232A. In embodiments including the secondary particulate filter 232B, for example, the secondary particulate filter 232B can include a polyester air filter having a pore size in a range of 0.1 μm to 10 μm, which prevents particulates of a size greater than about 100 nm (or greater) from passing through the secondary particulate filter 232B.

As illustrated in the diagram of FIG. 2C, the motor unit of the suction and collection assembly 245 is positioned within the airflow pathway 270 after the second filter 232. In this example, the motor unit includes a fan motor that pulls air from a first end of the motor unit and forces the air away from a second end of the motor unit. Also, in this example, one or more walls and/or structures can be used to assist in the direction of airflow.

In some embodiments, for example, the particulate filter unit 230 includes a third filter 233, comprising one or more air purifying filters 233A and/or 233B, which is positioned after the first filter 231 (and, in this example, the second filter 232) along the airflow pathway 270. The third filter 233 is configured to purify the air and filter out any remaining particulates, as well as moisture, that may have passed through the first filter 231 (and/or second filter 232). In embodiments including the air purifying filter 233A, for example, the air purifying filter 233A can include a high-efficiency particulate air (HEPA) filter having a pore size in a range of 20 nm to 300 nm, which prevents particular miniscule, yet harmful particulates from passing through, e.g., including but not limited to small-size pollen, dirt, dust, moisture, bacteria (e.g., which can range from 0.2 to 2.0 μm), virus (e.g., from 0.02 to 0.3 μm), and submicron liquid aerosol (e.g., from 0.02 to 0.5 μm. In embodiments including the air purifying filter 233B, for example, the air purifying filter 233B can include an active carbon filter capable of filtering molecules within gases through one or more surfaces of activated carbon (e.g., charcoal) disposed within the active carbon filter housing. The active carbon filter is configured to screen volatile organic compounds (VOCs) that may have been present within the sterilized particulate-containing air 201B, as well as remove odors from the air, e.g., by facilitating chemical reactions with such reactants on the filter surface.

Some microbes, e.g., bacteria (e.g., which can have a size of 0.2 μm to 5.0 μm) and viruses (e.g., which can have a size from 20 nm to 500 nm), may not be capturable by the filters of a vacuum cleaner. As such, the UV light unit 210 of the air purification system 200, positioned before the filters of the vacuum cleaner 102, is configured to harm or kill such microbes, rendering their structures innocuous should they cycle through the vacuum cleaner 102. Yet, notably, in some embodiments, the UV light unit 210 can include a second UV light unit 210 (not shown) positioned after the first filter 231 or the third UV light unit 210, to emit UV light that harms and/or kills such microbes that are not captured by the prior filter unit(s). For example, the second UV light unit 210 includes a second set of UV light emitters 212 (secured by housing or frame structure 211) that is additionally positioned within the chamber of the housing of the motor unit and third filter 233, e.g., prior to one or both of the example HEPA filter 233A or active carbon filter 233B.

In some embodiments of the air purification system 200, the system provides a tripartite filtration system, comprising UV light emitter(s), a HEPA filter, and an active carbon (e.g., charcoal) filter, integrated in certain regions and units of a vacuum cleaner in a manner that enables the operation of the individual subunits of the air purification system, e.g., the UV light unit 210 and the particulate filter unit 230. For example, based on the speed of airflow (suction) by the motor unit, the subsystems of the air purification system 200 are incorporated in their particular locations along the airflow pathway 270 to operate effectively.

In some embodiments of the air purification system 200, the system includes a scent system that includes a material with a volatile chemical substance that disperses in the purified output air 201C. For example, the scent system can include scent pods or packs that are disposed in one or more locations of the airflow pathway 270, e.g., including but not limited to proximate the first filter 231 (e.g., positioned by the example mesh filter), proximate the second filter 232 (e.g., positioned by to the example polyester filter), and/or proximate the third filter 233 (e.g., positioned by the example HEPA filter or example active carbon filter). In some implementations, the scent system includes a scent pod or pack that is placed after the third filter proximate the exhaust 219, such that the volatile scented substance(s) is dispersed within the sterilized, filtered, and purified output air 201C as it is forced out of the vacuum cleaner 102. In some embodiments, the example scent pod or pack can be easily replaceable by inserting and removing the pod or pack in an interior compartment, e.g., accessible via the exterior of the vacuum cleaner. Examples of the scent system are shown in FIGS. 2E and 2F.

FIG. 2E shows a diagram of an example embodiment of a scent system, including one or more scent pods and/or packs, for a vacuum cleaner, in accordance with the present technology. In some embodiments, for example, the vacuum cleaner 102 can include a scent pod and/or pack 247 disposed in or at the example first filter 231 (e.g., mesh filter) of the particulate filter unit 230 that excludes the dirt, dust, etc. particulates of a certain size. The location of the scent pod and/or pack 247 coupled to or proximate with the first filter 231 can optimize the scent distribution in the air distributed within and outside of the vacuum cleaner 102 since, typically, air flow along the airflow pathway 270 can be at its highest speeds (e.g., when air swirls to separate the dirt, dust, etc. particulates), promoting greater diffusion of the volatile scented substance(s) in the airflow. Some example embodiments of the scent pod or pack 247 can include a conical shape or be configured to interface with a conically shaped first filter 231, illustrated in FIG. 2F.

FIG. 2F shows a diagram 241 of example embodiments of the scent pods and/or packs 247 configured at the first filter 231. Examples of the scent pods and/or packs 247 are illustrated as “scent stoppers 247S,” where the scent pods and/or packs 247 are configured in a hemispherical-, cylindrical-, semi-conical-, or dome-shaped (or other-shaped) scent storage/delivery structure 247S3 disposed underneath an upper base 247S1 with a handle portion 247S2, which allows the scent structure 247S3 of the scent stopper 247S to be inserted in an upper region of the example cone mesh filter 231F (with the upper base 247S1 resting on a top wall of the cone mesh filter 231F) and removed (e.g., for replacement) by use of the handle portion 247S2. Similar examples of the scent pods and/or packs 247 are illustrated as “scent pods 247P,” where the scent pods and/or packs 247 are configured in an insertable inverse cone or array of projections as the scent storage/delivery structure 247P3 disposed underneath an upper base 247P1 with a handle portion 247P2, which allows the scent structure 247P3 of the scent pod 247P to be inserted in an upper region of the example cone mesh filter 231F (with the upper base 247S1 resting on a top wall of the cone mesh filter 231F) and removed (e.g., for replacement) by use of the handle portion 247P2.

Also shown in FIG. 2F is a diagram 242 depicting an example housing of the particulate filter unit 230, wherein example embodiments of the scent pods and/or packs 247 (e.g., such as the scent stoppers 247S or the scent pods 247P) can be attached to an inside surface of a lid of the housing. For instance, when the lid is closed, the example scent pods and/or scent packs 247 sits in a swirl zone during operations of the vacuum cleaner, distributing scented sub stance(s) in the airflow.

Referring back to FIG. 2E, in some embodiments, for example, the vacuum cleaner 102 can also or alternatively include a scent pod and/or pack 248 disposed at or proximate to the second filter 232 (e.g., one or more secondary particulate filters 232A and/or 232B) of the particulate filter unit 230. Moreover, for example, the vacuum cleaner 102 can also or alternatively include a scent pod and/or pack 249 disposed at or proximate to the third filter 233 (e.g., example HEPA filter or example active carbon filter) of the particulate filter unit 230.

FIG. 3 shows a diagram of another example embodiment of the vacuum cleaner 100, shown as vacuum cleaner 302, that includes an example embodiment of the air purification system 200, in accordance with the present technology. In some embodiments, the vacuum cleaner 302 includes an upper assembly 305 connected to a lower assembly 310, e.g., via a joint 315. The upper assembly 305 includes a handle portion 325 positioned for a user to grasp during operation of the vacuum cleaner 302. For example, the upper assembly 305 can pivot, rotate, or otherwise move relative to the lower assembly 310 to facilitate a user's operation of the vacuum cleaner 302, such as pushing and steering the lower assembly 310 along the surface to be cleaned using the upper assembly 305. The vacuum cleaner 302 includes a hose 371 operably coupled between the lower assembly 310 and the upper assembly 305. In some embodiments, the vacuum cleaner 302 includes a power cord for connecting the vacuum cleaner 302 to an external power source. In some embodiments, the vacuum cleaner 302 includes electronic components, including, but not limited to, a power converter (e.g., AC/DC conversion) and/or a power supply (e.g., a battery).

The lower assembly 310 can include one or more motility components (e.g., wheels or tracks) to facilitate travel of the lower assembly 310 along the surface. In some embodiments, the motility component(s) can include powered motility units, such as motorized wheels. In other embodiments, the motility component(s) can be unpowered (such as freewheeling or otherwise freely movable), except for the pushing force provided by a user upon the vacuum cleaner 302. In some embodiments, such as the example shown in FIG. 3, the motility component(s) can be encased, at least partially, within a housing 311 of the lower assembly 310. The lower assembly 310 can include a suction input 335, which receives a suction airflow to pull debris (such as waste or particles) from the surface. The suction input 335 can, in some embodiments, include a plurality of inputs disposed in one or more positions of the lower assembly 310. For instance, in some embodiments, the lower assembly 310 includes a main suction input located on or along a bottom region of the housing 311. In some embodiments, for example, the suction input 335 can include the main suction input and one or more corner suction units 336 that are positioned in one or both front corners of the housing 311 and/or in one or both rear corners of the housing 311. Further examples pertaining to example embodiments of a vacuum cleaner comprising one or more corner suction units are disclosed in U.S. patent application Ser. No. 17/813,277, titled “Corner Suction Devices for Vacuum Cleaners, and Associated Systems and Methods,” and filed Jul. 18, 2022, which the entire contents are incorporated by reference as part of the disclosure of this patent document and all purposes.

The lower assembly 310 can include a brush disposed on an underside of the housing 311 (not shown) to agitate debris on the surface (e.g., to facilitate release of such debris) to promote suction of the debris into the suction input 335. In some embodiments, the brush is motorized such that it rotates against the surface. In some embodiments, the brush can be switched between an engaged and disengaged position, where the brush is configured to rotate in the engaged position and not rotate in the disengaged position.

The vacuum cleaner 302 can include a suction and collection unit 345, which, in the example of FIG. 3, is disposed in the upper assembly 305, to facilitate providing suction to the suction input 335 and collection of dirt, dust, microbes, allergens, and other particles (“debris”) from the suction input 335. In various embodiments, for example, the suction and collection unit 345 can include: a suction drive unit 350 (e.g., a motor, fan, etc.) operatively connected to the suction input 335 to provide the suction airflow; a filtration unit 355 (e.g., cyclone filtration system and/or particulate filter unit) operatively connected between the suction input 335 and the suction drive unit 350 to remove debris 361 from the suction airflow passing from the suction input 335 to the suction drive unit 350; and/or a debris collection chamber 360 (e.g., debris collection canister) operatively connected to the filtration unit 355 to collect the debris 361 that is filtered from the suction airflow. The debris-filtered air 362 is exhausted out of the vacuum cleaner 302, e.g., via positive pressure and/or secondary air flow system. The suction and collection unit 345 can be connected to the suction input 335 via an airflow pathway 370. For example, the airflow pathway 370 can connect the suction input 335 to the filtration unit 355 and suction drive unit 350. In some embodiments, the suction drive unit 350 includes a motor, fan, or other suitable source of airflow suction. The debris collection chamber 360 can be removable and replaceable from the vacuum cleaner 302 to facilitate emptying the debris collection chamber 360.

The example vacuum cleaner 302 shown in FIG. 3 depicts some or all of the components of the suction and collection unit 345 disposed in the upper assembly 305, e.g., encased by a base, housing, or frame 375. Whereas, in some embodiments of the vacuum cleaner 302, some or all of the components of the suction and collection unit 145 can be disposed in the lower assembly 310. In such embodiments, the lower assembly 310 and the suction and collection unit 345 can forma lower vacuum unit that can be operable independently of the upper assembly 305 and the handle portion 325. In some embodiments, an independent lower vacuum unit can be configured as a robotic vacuum unit. Further examples pertaining to example embodiments of a robotic vacuum cleaner as an example lower vacuum unit for the vacuum cleaner 302 are disclosed in U.S. patent application Ser. No. 17/813,292, titled “Modular Vacuum Cleaners,” filed Jul. 18, 2022, and which the entire contents are incorporated by reference as part of the disclosure of this patent document and all purposes.

For example, in some embodiments, the joint can facilitate separation of the upper assembly 305 from the lower assembly 310. In some embodiments, the airflow pathway 370 can include a hose that is detachable from the lower assembly 310, in which the detachable end of the hose can allow for attachment pieces (not shown), e.g., such as a pet hair brush, dusty brush, crevice tool, etc., to attach to the hose's detachable end and allow a user to vacuum clean surfaces difficult or otherwise unreachable by the lower assembly 310.

The vacuum cleaner 302 includes an example embodiment of the air purification system 200. In this example, the air purification system 200 is employed, at least partly, after the suction drive unit 350 in the airflow pathway 370. In some embodiments, for example, the air purification system 200 employed in the example vacuum cleaner 302 (shown in FIG. 3) includes an embodiment of the UV light unit 210 to emit UV light at the air flowed through the system 200. In some embodiments, for example, the air purification system 200 employed in the example vacuum cleaner 302 includes an embodiment of the tripartite filtration system, comprising UV light emitter(s), a HEPA filter, and an active carbon (e.g., charcoal) filter.

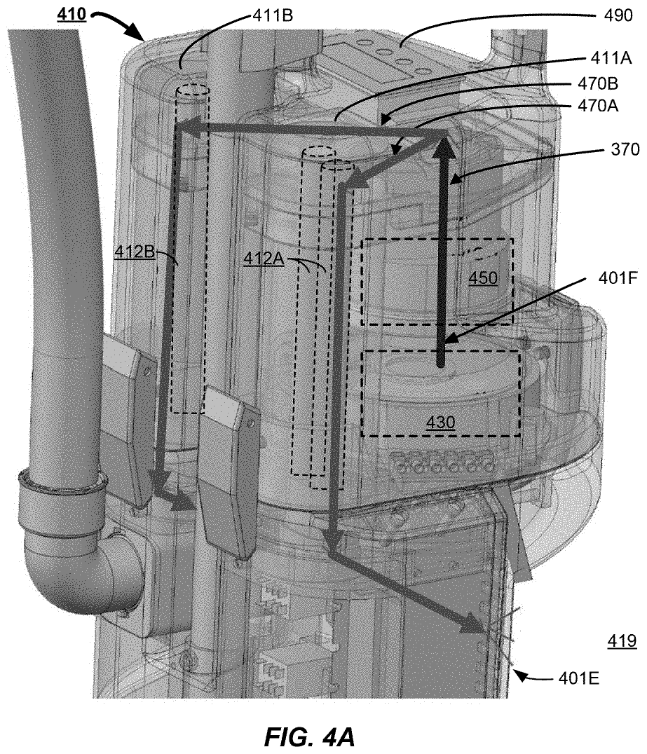

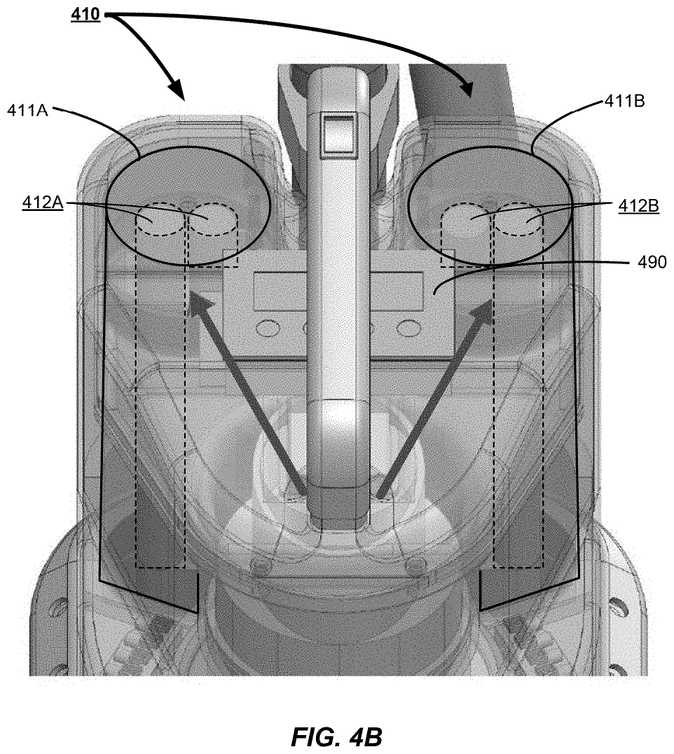

FIGS. 4A and 4B show diagrams of an example embodiment of the UV light unit 210, labeled 410, is disposed in the air purification system 200 post-debris filtration (e.g., at least after some debris filtration/separation) to sanitize filtered air or exhaust air, in accordance with the present technology. As shown in the examples of FIGS. 4A and 4B, the UV light unit 410 is disposed in an example embodiment of the vacuum cleaner 302. The diagram of FIG. 4A illustrates a side perspective view of the example UV light unit 410, and the diagram of FIG. 4B shows the example UV light unit 410 from a front perspective view.

The UV light unit 410 includes two separate UV chamber structures 411A and 411B that secures a respective set of one or more UV light emitters 412A and 412B to emit UV light as rays to sterilize air containing microbes (of the, at least partially, filtered air 401F) as it flows within a UV light emission zone (contained within the UV chamber structures 411A and 411B) as part of the airflow pathway 370 through the vacuum cleaner (e.g., vacuum cleaner 302) that expels sterilized exhaust air 401E out of exhaust 419 of the vacuum cleaner. While the example of FIGS. 4A and 4B show two separate UV chamber structures 411A and 411B with corresponding UV light emitters 412A and 412B, respectively, it is understood that the UV light unit 410 can include two or more UV light systems. Also illustrated in the diagram of FIG. 4A is an example of the suction drive unit 150, labeled as vacuum motor 450, disposed after one or more filters of a particulate filter unit 430 (e.g., which can include a HEPA filter), which provides the suction force for creating the air flow to drive the air in the vacuum cleaner along the airflow pathway 370.

In the example shown, the UV light unit 410 includes the separate UV chamber structures 411A and 411B to increase the UV radiation exposure and intensity received by the vacuumed air, where a single air flow airstream in the airflow pathway 370 splits into two separate air streams 470A and 470B directed to UV chamber structures 411A and 411B, each with its own UV light bulb(s). This configuration increases the total volume of air that is exposed to the radiation while preventing the UV light unit 410 from requiring a larger, single UV light bulb, e.g., which can save on significant design space in the vacuum cleaner, as well as save on cost associated with larger single UV light bulbs. For example, the emitted UV light can be more efficiently transmitted within the respective chamber structures 411A and 411B to result in enhanced neutralization of the germs (e.g., relative to a single UV light chamber) in the respective air streams 470A and 470B. For example, an increased amount of germs can be sterilized by the example UV light unit 410 configured in the separate UV chamber structures 411A and 411B and respective UV light emitters 412A and 412, e.g., based a reduced volume and/or distance of the target germs from the UV light source (e.g., UV light emitters 412A and 412B, respectively), e.g., thereby increasing the effective power of the UV light and time of exposure to UV light for a given unit of energy required to power the UV light source in emission of the UV light.

In some embodiments, the UV light unit 410 includes a control unit 490 to adjust one or more settings of the UV light to be emitted by the UV light unit 410. In the example shown in FIGS. 4A and 4B, the control unit 490 can include a processor and memory inside a housing, which can be positioned on an exterior of the vacuum cleaner (e.g., vacuum cleaner 302). In some embodiments, for example, the control unit 490 include a display screen (e.g., providing a graphic user interface (GUI) display to allow output and input of information to the control unit). In some embodiments, for example, the control unit 490 can include a wireless transceiver to transmit and receive data between an external computing device (e.g., a smartphone, tablet, laptop, desktop, smart-wearable device like a smartwatch, smartglasses, etc., and/or server in the cloud), where the data transmissions can include instructions for the control unit 490 to execute to modify a setting of the UV light unit 410. Example settings can include, but are not limited to, a value of light emission intensity and/or pulse frequency of the UV light, a number of UV light emitting elements (e.g., UV light bulbs) among the UV light emitters 412A and/or 412B, or an airflow actuator-router to open or close a valve in the airflow passages corresponding to the two separate air streams 470A and 470B to direct the filtered air 401F through one or more selected passages to direct the filtered air 401F to a selected one or group of the UV chamber structures 411A and 411B.

In some implementations, for example, each of the UV light emitters 412A and 412B of the UV light unit 410 can include a separate (set of) electrical switches to allow individual control of the UV light bulbs, so as to help prevent a user from accessing any of the UV light bulbs when powered on and thereby prevent the user from being exposed to the UV light. In example embodiments of the UV light unit 410, the UV chamber structures 411A and 411B are lined with material to reflect the UV light back into the center of the chamber. For example, this prevents any of it from bleeding through a plastic material of the vacuum cleaner housing; and this also prevents any UV light from escaping the UV chamber, e.g., such as through an exhaust port. In this manner, the UV light unit 410 is capable of preventing a user from experiencing any UV exposure at any time when using the vacuum cleaner.

In some embodiments of the vacuum cleaner 302, such as in the example shown in FIG. 3, the vacuum cleaner 302 includes a sealed system to prevent minuscule particles from leaching out of the airflow pathway 370. For instance, particles of all sizes picked up by the vacuum cleaner need to be fully trapped and sealed inside the debris collection canister (dustbin) or the filters of the vacuum cleaner, otherwise they can mix into the outside air of a room. The sealed system in some embodiments of the vacuum cleaner 302 includes a mechanical design comprising a plurality of elastomer gaskets and/or O-rings to seal the joints for the vacuum pressure side of the airpath (e.g., from the cleaning surface, through the suction input 335 to the filtration unit 355 and up to the suction drive unit 350).

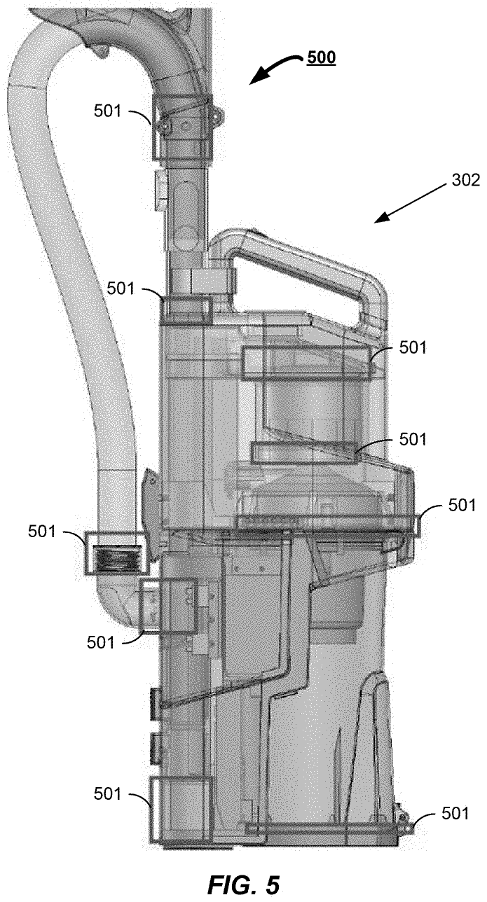

FIG. 5 shows a diagram illustrating locations of an example vacuum cleaner employing an example embodiment of a sealed system 500 to prevent leaks of dirty air, in accordance with the present technology. The diagram depicting a portion of the vacuum cleaner 302 from FIG. 3 that shows locations 501 where plurality of elastomer gaskets and/or O-rings are positioned between two or more joined materials or structures of the vacuum cleaner in the airpath, thereby sealing these locations to prevent leaks.

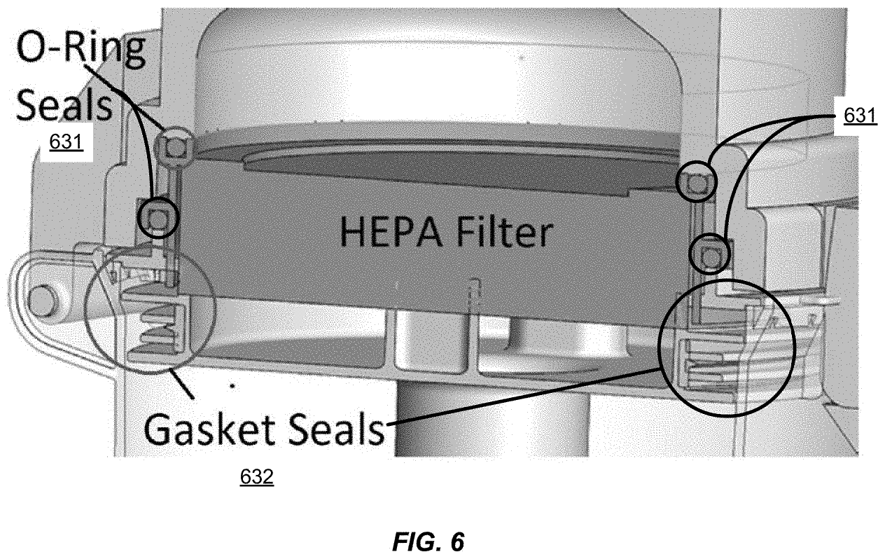

FIG. 6 shows a diagram illustrating an example embodiment of the particulate filter unit 430 shown in the example vacuum cleaner depicted in FIG. 4A that incorporates an embodiment of the sealed system 500. In some embodiments, the example filter unit 430 can include a HEPA filter and a plurality of O-rings 631 and/or gasket seals 632 at the sealing locations, e.g., just before and surrounding the example HEPA filter. For example, these locations are where the largest accumulation of small-particle debris can combine with the largest airflow impedance of the filter. This combination makes this the most difficult sealing location because the debris will follow the path of least resistance in terms of air pressure. The example sealing materials (e.g., O-ring(s) 631 and/or gasket(s) 632) placed in these locations provide more resistance to airflow than the HEPA filter to force the air through the filter and prevent any leaks.

In some implementations, the sealing system 500 is only employed between the connecting components of the vacuum cleaner that are positioned prior to the suction drive unit 350 and/or the air purification system 200, after which air may already been cleaned and/or sanitized.

In some embodiments of the vacuum cleaner 302, such as in the example shown in FIG. 3, the vacuum cleaner 302 includes a low airflow sensing system. Low airflow in a vacuum cleaner can contribute to poor air quality for a variety of reasons, such as a bad filter or filter connection, a full filter, and/or a clog in the airflow passageway. For example, low airflow can be caused by a filter that needs to be replaced, which means that the vacuum cleaner is no longer effectively filtering contaminants out of its airstream and instead is ejecting them out of the exhaust port. Also, for example, a clog anywhere in the airpath will cause high pressures and the potential for motor overheating. Excessively high pressures might exceed the sealed gaskets of the vacuum and cause air leaks. Overheating of the vacuum cleaner can cause failure of numerous parts that can all contribute to suboptimal air quality control.

In various embodiments, for example, the low airflow sensing system includes a sensor to detect a low airflow condition in communication with a processing unit (e.g., microcontroller, processor and memory, etc.) to evaluate the detected signal; and in some embodiments, for example, the low airflow sensing system includes an indicator unit in communication with the processing unit, such as an optical or audio indicator like an LED display or a speaker, respectively. In some embodiments, for example, the processing unit can be configured as an electrical circuit, which in some embodiments of the electrical circuit, the electrical circuit can include a thermistor electrically coupled to a power source (e.g., battery) and circuit components (e.g., transistors, resistors, op amps, capacitors, etc.), which can be coupled to an LED to optically indicate the determination of the low airflow condition.

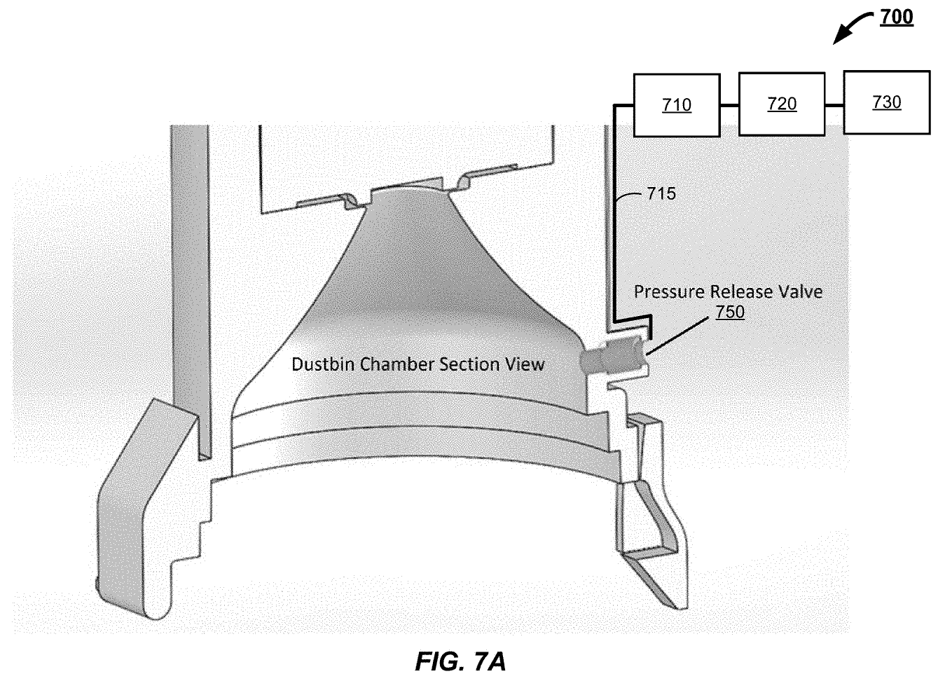

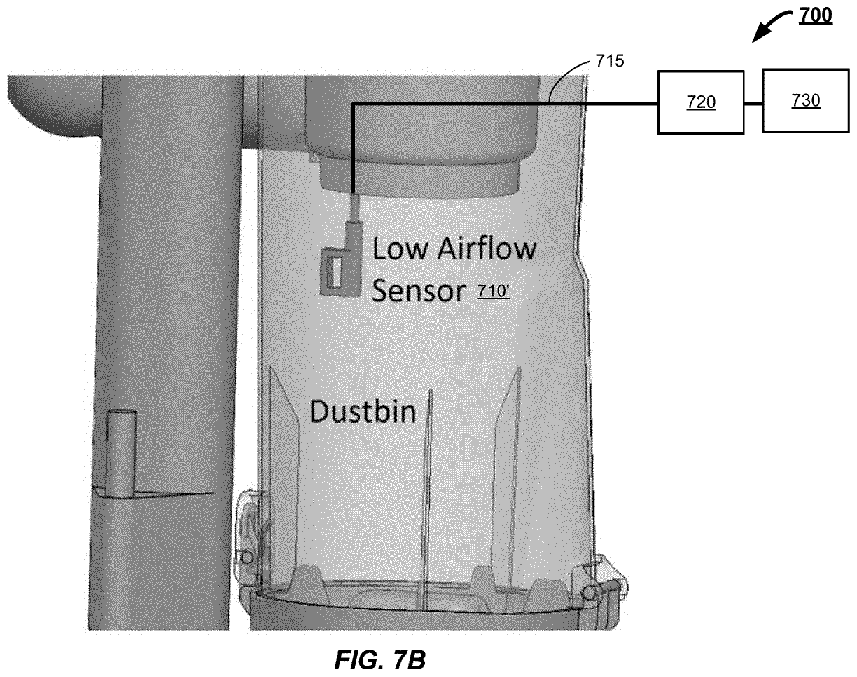

FIG. 7A shows a diagram illustrating an example embodiment of a low airflow sensing system 700, in accordance with the present technology. In some embodiments, for example, the low airflow sensing system 700 includes a sensor 710 that is configured to interact with a pressure release valve 750 that is built into the vacuum cleaner. The low airflow sensing system 700 includes a processing unit 720, in communication with the sensor 710, and an indicator unit 730, in communication with the processing unit 720. In general, conventional vacuum cleaners include at least one pressure release valve in the airflow passage since an air pressure release valve is a safety mechanism that is pushed open by excess pressure to allow air to escape in the event to a clog or airflow blockage. As such, the example sensor 710 of the low airflow sensing system 700 includes electrical contacts 715 interfaced with the pressure release valve 750 to detect when the pressure release valve 750 has been triggered. When the electrical signal triggered by the pressure release valve 750, the low airflow sensing system 700 can produce a control signal to activate the indicator unit, e.g., light up an indicator LED to alert a user of low airflow so they can take corrective action.

A full canister is simply another type of low-airflow clog that stresses the filters and gaskets in the same way as other clogs. In implementations of the example low airflow sensing system 700, a user can be informed (e.g., via the indicator) and thereby know when the user needs to empty the dustbin, e.g., allowing him/her to use the vacuum cleaner without needing to check, guess, or worry about forgetting to empty the bin. As discussed above, when clogs occur, overheating of the vacuum cleaner can occur. In some embodiments of the low airflow sensing system 700 can include a temperature sensor integrated in a portion of the dustbin canister.

FIG. 7B shows a diagram illustrating an example embodiment of a low airflow sensing system 700B, in accordance with the present technology, that is disposed within a debris collection canister and can be used to indicate whether the canister is full (or near full). In some embodiments, for example, the low airflow sensing system 700B includes a temperature sensor 710′ disposed within or proximate to an airflow passage of a debris collection canister (dustbin) to detect a temperature, where the processing unit 720 is configured to determine if the detected temperature is above a threshold, indicating a clog. In various embodiments, for example, the example low airflow sensing system 700B can include some or all of the components of the example low airflow sensing system 700. In some embodiments of the low airflow sensing system 700B, for example, the sensor 710′ is a temperature probe located at the top of the dustbin. For example, if the dustbin is full, airflow (and thus heat removal) over the sensor will sharply decrease, triggering a full canister indicator light of the indicator unit 730. In this manner, the low airflow sensing system 700B can be implemented as a full-canister sensor/detector.

For example, during normal operation with a dustbin that is not yet full, air will be flowing by the temperature probe at a predictable rate. This airflow takes heat away from the temperature probe. The rate of heat loss can be used to accurately calculate the velocity of the air passing over the temperature probe. If the dustbin becomes full, then the airflow at the top of the dustbin (where the example temperature probe sensor 710′ is located) will drastically decrease, although not necessarily cause a high-pressure clog to trigger the low-airflow sensor. This decrease in airflow (and thus heat removal) is detected by the temperature probe, and the processing unit 720 (e.g., an electrical circuit) then sends a signal to the indicator unit 730 (e.g., to light up an indicator LED) to alert a user that the canister is full and should be emptied.

In some embodiments, the low airflow sensing system 700B can include a weight sensor (not shown) that can be disposed under the debris collection canister (dustbin), which can measure a weight of the canister on an underlying surface where the weight sensor is disposed, and thereby inform a control unit of the vacuum cleaner on a level of fullness of the debris in the dustbin, which can adversely affect the airflow in the vacuum cleaner.

In some embodiments of the vacuum cleaner 302, for example, the vacuum cleaner 302 can be configured as a hybrid upright floor-rolling—detachable lift-away vacuum cleaner. For instance, the suction and collection unit 345 of the vacuum cleaner 302 (e.g., the suction drive unit 350, the filtration unit 355, and/or the debris collection chamber, and/or optionally the air purification system 200) can be configured in a first (upper) portion of the housing 375 of the upper assembly 305 that is detachable from a second (lower) portion of the housing 375, e.g., via a latch mechanism, which allows the detachable portion to be separated from the handle 325 and the lower assembly 310. In this manner, for example, a user of the vacuum cleaner can carry the detachable portion of the vacuum in one hand and use the hose with the other hand to clean various locations that are difficult to reach with the full upright vacuum (e.g., stairs, corners, walls, etc.).

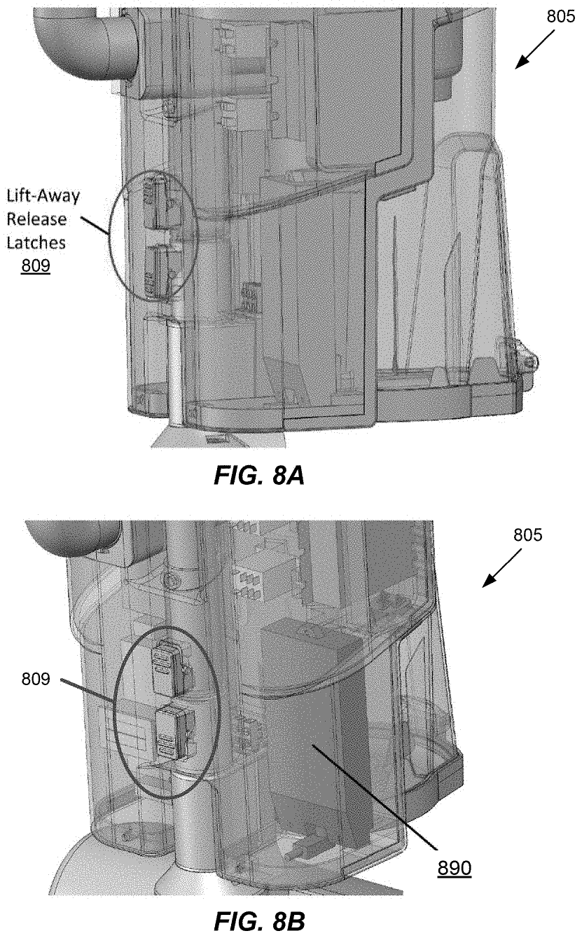

FIG. 8A shows a diagram illustrating (a partial view) of an example embodiment of the detachable portion, labeled 805, of an example hybrid upright floor-rolling detachable lift-away vacuum cleaner, in accordance with the present technology. In some implementations, for example, to use the detachable portion 805, which includes the suction and collection unit 345, a user simply presses on a pair of latches of a latching mechanism 809 to release the detachable portion 805 from the lower assembly 310 and the handle 325, and then subsequently lift the detachable portion 805 away to clean manually with the hose 371. In the example shown in FIG. 8A, the latching mechanism 809 includes two circled latches, where the latches reattach via torsion spring(s).

FIG. 8B shows a diagram depicting an example embodiment of a cordless, battery-powered configuration for the detachable portion 805. For example, the battery unit 890 of the detachable portion 805 can include one or more replaceable, rechargeable batteries to power the lift-away mode, e.g., disposed within an electronics compartment of the vacuum cleaner 302. For example, the battery unit 890 can be removed to be replaced or recharged; and a user can access the battery through a removable plastic panel next to the battery.

In various example embodiments of the vacuum cleaner 302 that includes the detachable portion 805, electric switches and circuitry can be configured (e.g., within the electronics compartment, proximate the battery unit 890) to detect when the detachable portion 805 has been lifted away and adjust the power source from a tethered power source (e.g., wall outlet via a power cord) to the battery unit 890. For example, logic gates within the vacuum's circuitry can lower the overall vacuum power usage during battery operation to optimize for battery life and runtime. In some implementations, for example, a user can activate switches at the top of the detachable portion 805 (e.g., such an example display of the control unit 490 shown in FIGS. 4A and 4B) to switch between “high” and “low” modes of power usage to meet whatever their preference is between greater cleaning performance (e.g., suction) and greater runtime.

### Examples

In some embodiments in accordance with the present technology (Example A1), an air purification system for a vacuum cleaner includes an ultraviolet (UV) light unit disposed in a first location within the vacuum cleaner along an airflow pathway, the UV light unit comprising a housing and one or more UV light emitters coupled to the housing, the UV light unit configured to emit UV light at air containing particles including dust, dirt, and microbes while the air containing the particles is flowing in the airflow pathway where the UV light unit is disposed, wherein emitted UV light is able to harm biological materials of the microbes to sterilize the air; and a particle filter unit disposed in a second location within the vacuum cleaner after the first location along the airflow pathway, the particle filter unit comprising one or both of a high-efficiency particulate air (HEPA) filter and an active carbon filter, the HEPA filter including a porous material to prevent at least some of the particles having a size greater than a pore size of the porous material from passing through the HEPA filter, and the active carbon filter including a securement structure that couples an activated carbon material having a chemically-reactive surface capable of filtering molecules within the air contacting the active carbon filter by facilitating chemical reactions with the molecules to remove from the air.

Example A2 includes the air purification system of any of examples A1-A10, wherein the UV light unit and particle filter unit are modular units that allow installation and removal at the first location and the second location within the airflow pathway of the vacuum cleaner, respectively.

Example A3 includes the air purification system of any of examples A1-A10, wherein the HEPA filter includes pores in a range of 20 nm to 300 nm.

Example A4 includes the air purification system of any of examples A1-A10, wherein the HEPA filter is operable to prevent particular small-size pollen, dirt, dust, moisture, bacteria, viruses, fungi, protists, and liquid aerosols from passing through the HEPA filter.

Example A5 includes the air purification system of any of examples A1-A10, wherein the active carbon filter includes charcoal.

Example A6 includes the air purification system of any of examples A1-A10, wherein the active carbon filter is configured to react with volatile organic compounds (VOCs) and remove odors from the air.

Example A7 includes the air purification system of any of examples A1-A10, wherein the UV light unit is in electrical communication with an electrical circuit of the vacuum cleaner that includes at least one of a power converter, power regulator, or power supply to provide electrical current to the one or more UV light emitters.

Example A8 includes the air purification system of example A7 or any of examples A1-A10, wherein the UV light unit includes a control unit, comprising a processor and memory, to provide control logic to operate the one or more UV light emitters.

Example A9 includes the air purification system of example A8 or any of examples A1-A10, wherein the control unit is configured to regulate one or more of an intensity, pulse frequency, or duration of the emitted UV light.

Example A10 includes the air purification system of any of examples A1-A9, further comprising a second UV light unit disposed in a third location within the vacuum cleaner along the airflow pathway, wherein the third location is after the second location in the airflow pathway, the second UV light unit comprising a housing and one or more UV light emitters coupled to the housing, the second UV light unit configured to emit UV light at filtered air that was at least partially filtered by the particle filter unit.

Example A11 includes the air purification system of any of examples A1-A10, wherein the air purification system includes one or more features of the air purification system of any of examples B1-B10.

In some embodiments in accordance with the present technology (Example B1), an air purification system for a vacuum cleaner includes an ultraviolet (UV) light unit disposed in a first location within the vacuum cleaner along an airflow pathway from a suction inlet to an exhaust outlet, the UV light unit comprising a first chamber and a second chamber and a first set of one or more UV light emitters and a second set of one or more UV light emitters positioned within the first chamber and the second chamber, respectively, wherein the UV light unit is configured to emit UV light via the one or more UV light emitters within the first chamber and the second chamber at air containing microbes while the air containing the microbes is flowing in the first chamber and the second chamber, wherein emitted UV light is able to harm biological materials of the microbes to sterilize the air; and a particle filter unit disposed in a second location within the vacuum cleaner positioned before the first location along the airflow pathway, the particle filter unit comprising one or both of a high-efficiency particulate air (HEPA) filter and an active carbon filter.

Example B2 includes the air purification system of any of examples B1-B10, wherein the first chamber is configured in a first airflow pathway that is separate from and parallel to a second airflow pathway, where the first airflow pathway and the second airflow pathway are split into separate air streams directed to the first chamber and the second chamber, respectively.

Example B3 includes the air purification system of any of examples B1-B10, wherein the first chamber and the second chamber are lined with a reflective material to reflect the emitted UV light to be contained within the respective chamber.

Example B4 includes the air purification system of any of examples B1-B10, wherein the UV light unit is in electrical communication with a control unit, comprising a processor and memory, to control one or more operations the one or more UV light emitters.

Example B5 includes the air purification system of example B4 or any of examples B1-B10, wherein the control unit is configured to regulate one or more of an intensity, pulse frequency, or duration of the emitted UV light.

Example B6 includes the air purification system of any of examples B1-B10, wherein the HEPA filter includes a porous material to prevent at least some of the particles having a size greater than a pore size of the porous material from passing through the HEPA filter.

Example B7 includes the air purification system of example B6 or any of examples B1-B10, wherein the HEPA filter includes pores in a range of 20 nm to 300 nm.

Example B8 includes the air purification system of any of examples B1-B10, wherein the active carbon filter includes a securement structure that couples an activated carbon material having a chemically-reactive surface capable of filtering molecules within the air contacting the active carbon filter by facilitating chemical reactions with the molecules to remove from the air.

Example B9 includes the air purification system of example B8 or any of examples B1-B10, wherein the active carbon filter includes charcoal and is configured to react with volatile organic compounds (VOCs) and remove odors from the air.

Example B10 includes the air purification system of any of examples B1-B10, further comprising a second UV light unit disposed in a third location within the vacuum cleaner along the airflow pathway, wherein the third location is before the second location in the airflow pathway, the second UV light unit comprising a housing and one or more UV light emitters coupled to the housing, the second UV light unit configured to emit UV light at unfiltered air containing the microbes and at least one of dust or dirt.

Example B11 includes the air purification system of any of examples B1-B10, wherein the air purification system includes one or more features of the air purification system of any of examples A1-A10.

The components in the drawings are not necessarily to scale and are not necessarily drawn consistently from one figure to another. Instead, emphasis is placed on clearly illustrating the principles of the present technology.

Implementations of the subject matter and the functional operations described in this patent document can be implemented in various systems, digital electronic circuitry, or in computer software, firmware, or hardware, including the structures disclosed in this specification and their structural equivalents, or in combinations of one or more of them. Implementations of the subject matter described in this specification can be implemented as one or more computer program products, i.e., one or more modules of computer program instructions encoded on a tangible and non-transitory computer readable medium for execution by, or to control the operation of, data processing apparatus. The computer readable medium can be a machine-readable storage device, a machine-readable storage substrate, a memory device, a composition of matter effecting a machine-readable propagated signal, or a combination of one or more of them. The term “data processing unit” or “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of nonvolatile memory, media and memory devices, including by way of example semiconductor memory devices, e.g., EPROM, EEPROM, and flash memory devices. The processor and the memory can be supplemented by, or incorporated in, special purpose logic circuitry.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a sub combination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

Only a few implementations and examples are described and other implementations, enhancements and variations can be made based on what is described and illustrated in this patent document.

What is claimed is:

1. An air purification system for a vacuum cleaner, comprising:
an ultraviolet (UV) light unit disposed in a first location within the vacuum cleaner along an airflow pathway, the UV light unit comprising a housing and one or more UV light emitters coupled to the housing, the UV light unit configured to emit UV light at air containing particles including dust, dirt, and microbes while the air containing the particles is flowing in the airflow pathway where the UV light unit is disposed, wherein emitted UV light is able to harm biological materials of the microbes to sterilize the air; and
a particle filter unit disposed in a second location within the vacuum cleaner after the first location along the airflow pathway, the particle filter unit comprising one or both of a high-efficiency particulate air (HEPA) filter and an active carbon filter, the HEPA filter including a porous material to prevent at least some of the particles having a size greater than a pore size of the porous material from passing through the HEPA filter, and the active carbon filter including a securement structure that couples an activated carbon material having a chemically-reactive surface capable of filtering molecules within the air contacting the active carbon filter by facilitating chemical reactions with the molecules to remove from the air.

2. The air purification system of claim 1, wherein the UV light unit and particle filter unit are modular units that allow installation and removal at the first location and the second location within the airflow pathway of the vacuum cleaner, respectively.

3. The air purification system of claim 1, wherein the HEPA filter includes pores in a range of 20 nm to 300 nm.

4. The air purification system of claim 1, wherein the HEPA filter is operable to prevent particular small-size pollen, dirt, dust, moisture, bacteria, viruses, fungi, protists, and liquid aerosols from passing through the HEPA filter.

5. The air purification system of claim 1, wherein the active carbon filter includes charcoal.

6. The air purification system of claim 1, wherein the active carbon filter is configured to react with volatile organic compounds (VOCs) and remove odors from the air.

7. The air purification system of claim 1, wherein the UV light unit is in electrical communication with an electrical circuit of the vacuum cleaner that includes at least one of a power converter, power regulator, or power supply to provide electrical current to the one or more UV light emitters.

8. The air purification system of claim 7, wherein the UV light unit includes a control unit, comprising a processor and memory, to provide control logic to operate the one or more UV light emitters.

9. The air purification system of claim 8, wherein the control unit is configured to regulate one or more of an intensity, pulse frequency, or duration of the emitted UV light.

10. The air purification system of claim 1, further comprising a second UV light unit disposed in a third location within the vacuum cleaner along the airflow pathway, wherein the third location is after the second location in the airflow pathway, the second UV light unit comprising a housing and one or more UV light emitters coupled to the housing, the second UV light unit configured to emit UV light at filtered air that was at least partially filtered by the particle filter unit.

11. An air purification system for a vacuum cleaner, comprising:
an ultraviolet (UV) light unit disposed in a first location within the vacuum cleaner along an airflow pathway from a suction inlet to an exhaust outlet, the UV light unit comprising a first chamber and a second chamber and a first set of one or more UV light emitters and a second set of one or more UV light emitters positioned within the first chamber and the second chamber, respectively, wherein the UV light unit is configured to emit UV light via the one or more UV light emitters within the first chamber and the second chamber at air containing microbes while the air containing the microbes is flowing in the first chamber and the second chamber, wherein emitted UV light is able to harm biological materials of the microbes to sterilize the air; and
a particle filter unit disposed in a second location within the vacuum cleaner positioned before the first location along the airflow pathway, the particle filter unit comprising one or both of a high-efficiency particulate air (HEPA) filter and an active carbon filter.

12. The air purification system of claim 11, wherein the first chamber is configured in a first airflow pathway that is separate from and parallel to a second airflow pathway, where the first airflow pathway and the second airflow pathway are split into separate air streams directed to the first chamber and the second chamber, respectively.

13. The air purification system of claim 11, wherein the first chamber and the second chamber are lined with a reflective material to reflect the emitted UV light to be contained within the respective chamber.

14. The air purification system of claim 11, wherein the UV light unit is in electrical communication with a control unit, comprising a processor and memory, to control one or more operations the one or more UV light emitters.

15. The air purification system of claim 14, wherein the control unit is configured to regulate one or more of an intensity, pulse frequency, or duration of the emitted UV light.

16. The air purification system of claim 11, wherein the HEPA filter includes a porous material to prevent at least some of the particles having a size greater than a pore size of the porous material from passing through the HEPA filter.

17. The air purification system of claim 16, wherein the HEPA filter includes pores in a range of 20 nm to 300 nm.

18. The air purification system of claim 11, wherein the active carbon filter includes a securement structure that couples an activated carbon material having a chemically-reactive surface capable of filtering molecules within the air contacting the active carbon filter by facilitating chemical reactions with the molecules to remove from the air.

19. The air purification system of claim 18, wherein the active carbon filter includes charcoal and is configured to react with volatile organic compounds (VOCs) and remove odors from the air.

20. The air purification system of claim 11, further comprising:
a second UV light unit disposed in a third location within the vacuum cleaner along the airflow pathway, wherein the third location is before the second location in the airflow pathway, the second UV light unit comprising a housing and one or more UV light emitters coupled to the housing, the second UV light unit configured to emit UV light at unfiltered air containing the microbes and at least one of dust or dirt.
